(19) United States
(12) Patent Application Publication
(10) Pub. No.: US 2016/0097106 A1
(43) Pub. Date: Apr. 7, 2016

(54) Methods and Systems for Using Probes in Conduits

(71) Applicants: Amelia C. Robinson, Houston, TX (US); Jennifer A. Hornemann, Englewood, CO (US)

(72) Inventors: Amelia C. Robinson, Houston, TX (US); Jennifer A. Hornemann, Englewood, CO (US)

(21) Appl. No.: 14/862,696

(22) Filed: Sep. 23, 2015

Related U.S. Application Data
(60) Provisional application No. 62/058,504, filed on Oct. 1, 2014.

(51) Int. Cl. C12Q 1/68; A01N 59/00

(52) U.S. Cl. CPC C12Q 1/6888; A01N 59/00

(57) ABSTRACT

The present disclosure relates to a method and system for identifying and treating corrosion within a conduit. The method involves using probes having a signal generator, and one or more corrosion mitigating chemical treatments such as biocides and/or corrosion inhibitors.

### CROSS REFERENCE TO RELATED APPLICATIONS

This application claims the priority benefit of U.S. patent application No. 62/058,504 filed Oct. 1, 2014 entitled METHODS AND SYSTEMS FOR USING PROBES IN CONDUITS, the entirety of which is incorporated by reference herein

### FIELD OF THE INVENTION

Embodiments of the disclosure relate to the field of petroleum exploration, development, and production. More particularly, embodiments of the disclosure relate to identifying and treating conduits to limit mechanism that chemically or biologically induce corrosion. For example, treating biologic material from planktonic or sessile organisms and their associated biofilms reduces the extent or development of film resulting in microbially induced corrosion in conduits.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

The exploration, development, and production of hydrocarbon from reservoirs has become increasingly challenging and costly. These assets tend to be more difficult to identify and evaluate. Production from marginal quality assets, unconventional reservoirs, drilling deep water wells in challenging environments, and transportation of over long distances is becoming increasingly more challenging. Further, the transportation of the hydrocarbons from these reservoirs is complex and costly. The transport of the produced fluids may include directing flow of hydrocarbons (e.g., gas and/or oil) along with water and other fluids or materials.

To transport these produced fluids, extensive networks of conduits (e.g., pipes, flowlines, gathering lines or pipelines) carry the produced fluids through various equipment and other petroleum processing devices. Reduced material integrity, as caused by corrosion, can cause conduits to become compromised or fail, impacting supply and delivery of the produced fluids as well as causing other impacts associated with loss of fluids from the conduit. Compromised material integrity can be a result of advanced aging of conduits or other modes, such as duration and chemical composition of produced fluids; life span of material components, changing fluid properties (i.e. increased water cut, decreased oil production), restricted access to remove solids, etc. Biologic and abiotic processes may be involved in corrosion along pipelines. These processes may include biologic material, such as biofilms and planktonic biological material. Biofilms are biologic material composed of an extracellular polymeric substance (EPS) that forms on conduit surfaces, while the planktonic biologic material are free-floating micro-organisms within the conduit. Linked biologic and chemical processes that occur within the conduit often result in corrosion. The corrosion can continue to advance until the conduits have to be replaced, which is a costly operational activity. This becomes even more problematic for non-piggable or deep water assets or where multiple fluids are blended at one common platform. Indeed, some of these facilities may not be designed to withstand certain compositional changes that may occur over time.

Typical treatments to inhibit corrosion include various chemical treatments such as biocides (e.g., chlorine) and corrosion inhibitors that may reduce microbial populations and corrosive components respectively in liquid carrying conduits. These chemical treatments are injected into the conduit and travel through the conduit mixing with other fluids being transported in the conduit. Conventional treatment processes require much higher volumes of chemicals to overcome retention of inhibitors (e.g., nearly 90%) and biocides in the oil phase, along with limited efficacy on biofilms particularly in non-piggable zones. In non-aqueous phase or by attachment or deactivation of fluid conditions (change in fluid chemistry) or phase transfer between non-aqueous and aqueous phases, emulsion or surfactant formation may result in biocide retention at the oil-water phase contact. Rough surfaces and removal of “protective” components within the biofilm, organisms are more likely exposed or lost from pipe surfaces. Microbial induced corrosion inhibitors may also turn off signals for microbes to attach to conduits and therefore remain planktonic. As a result, the mechanism of delivery ensures treatment where needed. The remaining fraction of the introduced biocide is generally only effective on planktonic organisms. Impact of planktonic organisms on corrosion is generally less, but the consumption of the biocide for their demise reduces the amount that is utilized on the sessile organisms and their associated biofilms. Further, while these biocides are flowing in the same conduit and may interact with the planktonic biological material, the flow pattern within the conduits may not provide a mechanism for the biocide to properly interact with the biologic material attached to the conduit's internal wall. That is, the injection of the biocides may merely disrupt biofilms, protective films, and fail to eliminate and/or reduce biofilms, and reduce action of biocides via metabolic versus other mechanisms.

Other methods may involve the use of a mechanical device (e.g., a pig) to remove the biologic materials from within the conduits. This approach may include launching the mechanical device into a conduit and having the mechanical device remove materials from within the conduit for specific sections of the flow path (e.g., pipeline or conduits). Yet, the mechanical device may be limited to mechanically remove biofilms that are in contact with the mechanical device. That is, biofilms that are housed inside pits or other sections of the conduit that are not in contact with the mechanical device may not be properly removed. Further, certain sections of the conduit may not be accessible for mechanical device operations due to bend geometry being too small for the mechanical device to pass through easily. The mechanical device does not necessarily remove all of the biofilm material.

Accordingly, there exists a need for reliable, reproducible and efficient means for identifying and managing corrosion in conduits, particularly those derived from biologic materials. In particular, there exists a substantial need for an efficient and cost effective identification and treatment of biologic materials and limiting their initiation and proliferation in pipelines.

Other material may be found in the following references: Marsh et al., “Pipeline Internal Corrosion Assessment, Fitness for Purpose and Future Life Prediction”, NACE Corrosion Conference p. 1 to 5 (2010); Sooknah et al., “Monitoring Microbiologically Influenced Corrosion: Review of Techniques”, NACE Corrosion Conference, p. 1 to 17 (2007); Raman et al., “Evaluation of effective biocides for SRB to control microbiologically influenced corrosion”, Materials and Corrosion, vol. 59, No. 4, p. 329 to 334 (2008); Slowing et al., “Mesoporous Silica Nanoparticles for Drug Delivery and Biosensing Applications”, Advanced Functional Materials, vol. 17 p. 1225 to 1236 (2007); Kumar, “Amino-functionalized graphene quantum dots: origin of tunable heterogeneous photoluminescence”, Royal Society of Chemistry, p. 1 to 8 (2014); and Epand et al., “Bacterial membrane lipids in the action of antimicrobial agents”, Journal of Peptide, vol. 17, p. 298 to 305 (2011).

### SUMMARY

In one embodiment, a method for identifying and treating biologic materials is described. The method includes providing a probe composition comprising one or more probes (e.g., nanoprobes); wherein each of the one or more probes comprises: a tag; and one or more of a signal generator and a chemical treatment, wherein the probe is configured to generate a signal when the tag associates with a target biologic material, if the signal generator is present in the probe, and release the chemical treatment when the tag associates with the target biologic material, if the chemical treatment is present in the probe; releasing the probes into a conduit; if the probe composition includes one or more probes having the signal generator, detecting the presence of a signal generated by the signal generator on association of the tag with the target biologic material.

In another embodiment, a probe composition is described. The probe composition includes one or more probes, wherein the probe (e.g., a nanoprobe) comprises: at least one tag capable of associating with a target biologic material; and one or more of a signal generator and a chemical treatment, wherein the probe is configured to (i) generate a signal when the tag associates with a target biologic material, if a signal generator is present in the probe, and release the chemical treatment when the tag associates with the target biologic material, if a chemical treatment is present in the probe.

In still another embodiment, a method of identifying and treating biologic materials within a conduit is described. The method includes providing a first probe (e.g., first nanoprobe); wherein the first probe comprises: a first tag that associate with a first target biologic material; and one or more of a first signal generator and a first chemical treatment, wherein the first probe is configured to generate a first signal when the first tag associates with the first target biologic material, if the first signal generator is present in the first probe, and release the first chemical treatment when the first tag associates with the first target biologic material, if the first chemical treatment is present in the first probe; providing a second probe (e.g., second nanoprobe); wherein the second probe comprises: a second tag that associate with a second target biologic material; and one or more of a second signal generator and a second chemical treatment, wherein the second probe is configured to generate a second signal when the second tag associates with the second target biologic material, if the second signal generator is present in the second probe, and release the second chemical treatment when the second tag associates with the second target biologic material, if the second chemical treatment is present in the second probe; releasing a probe composition comprising the first probe and the second probe into a conduit; if the probe composition includes a first probe having the first signal generator, measuring the first signal, wherein the first signal is generated by the first signal generator on association of the first tag with the first target biologic material; and if the probe composition includes a second probe having the second signal generator, measuring the second signal, wherein the second signal is generated by the second signal generator on association of the second tag with the second target biologic material.

In yet another embodiment, a system for the identifying and treating biologic materials or corrosive environment within a conduit is described. The system includes a delivery device configured to store a probe composition comprising one or more probes. Each of the one or more probes comprises a tag capable of associating with a target biologic material or corrosive environment; and one or more of a signal generator, and a chemical treatment, wherein the probe is configured to generate a signal when the tag associates with a target biologic material or a corrosive environment. For example, if a signal generator is present in the probe, release the chemical treatment when the tag associates with the target biologic material, if a chemical treatment is present in the probe. Additionally, release of a chemical treatment when the tag associates with the target corrosive environment, if a chemical treatment is present in the probe. The system also includes at least one detector capable of monitoring the interaction of the target biologic materials or corrosive environment with the one or more probes. The one or more probes may be nanoprobes.

In yet another embodiment, the method for the identifying and treating a corrosive environment within a conduit is described. The method may include providing a probe composition comprising one or more probes (e.g., nanoprobes); wherein each of the one or more probes comprises: a tag; and one or more of a signal generator (e.g., nanoparticle or inorganic fluorophore) and a chemical treatment, wherein the probe is configured to generate a signal when the tag associates with a target corrosive environment, if the signal generator is present in the probe, and release the chemical treatment when the tag associates with the target corrosive environment, if the chemical treatment is present in the probe; releasing the probes into a conduit; if the probe composition includes one or more probes having the signal generator, detecting the presence of a signal generated by the signal generator on association of the tag with the target corrosive environment. The tag may be an electrochemical tracer, redox sensitive tracer, reactive functional tracer, anode potential tracer, cathode potential tracer and/or the like.

In still yet another embodiment, the method for the identifying and treating target materials or environments within a conduit is described. The method may include providing a probe composition comprising one or more probes. Each of the one or more probes comprises: a tag; and one or more of a signal generator, a, chemical and a corrosion inhibitor, wherein a target material comprises one or more of a biological material and a corrosive environment and wherein the probe is configured to generate a signal when the tag associates with a target material, if the signal generator is present in the probe, release the biocide when the tag associates with the target material, if the biocide is present in the probe, and release the corrosion inhibitor when the tag associates with the target material, if the inhibitor is present in the probe. Then, the probes may be released into a conduit. If the probe composition includes one or more probes having the signal generator, the method may detect the presence of a signal generated by the signal generator on association of the tag with the target material. Further, the probes may include a combination of different probes that are each configured to interact with a different target material.

### BRIEF DESCRIPTION OF THE DRAWINGS

The foregoing and other advantages of the present disclosure may become apparent upon reviewing the following detailed description and drawings of non-limiting examples of embodiments.

FIG. 1 is a flow chart for identifying and treating biologic materials in accordance with an exemplary embodiment of the present techniques.

FIGS. 2A, 2B, 2C and 2D are diagrams of identification probes in accordance with an exemplary embodiment of the present techniques.

FIGS. 3A, 3B and 3C are diagrams of treatment probes in accordance with an exemplary embodiment of the present techniques.

FIGS. 4A, 4B and 4C are diagrams of encapsulated probe systems in accordance with an exemplary embodiment of the present techniques.

FIGS. 5A, 5B, 5C, 5D, 5E, 5F, and 5G are diagrams of the use of the probes for a conduit in accordance with an exemplary embodiment of the present techniques.

FIG. 6 is a diagram of a monitoring tool for use with probes in a conduit in accordance with an exemplary embodiment of the present techniques.

FIG. 7 is a block diagram of a computer system that may be used to perform any of the methods disclosed herein.

### DETAILED DESCRIPTION

In the following detailed description section, the specific embodiments of the present disclosure are described in connection with preferred embodiments. However, to the extent that the following description is specific to a particular embodiment or a particular use of the present disclosure, this is intended to be for exemplary purposes only and simply provides a description of the exemplary embodiments. Accordingly, the disclosure is not limited to the specific embodiments described below, but rather, it includes all alternatives, modifications, and equivalents falling within the true spirit and scope of the appended claims.

Various terms as used herein are defined below. To the extent a term used in a claim is not defined below, it should be given the broadest definition persons in the pertinent art have given that term as reflected in at least one printed publication or issued patent.

“Conduit”, as referred herein, refers to any tubular member through which fluids are conveyed. The conduit is a general conveyor of liquids and/or gases, which may be in multiple phases. The conduit may include one or more of pipes, pipelines, flowlines, tubulars, tubing, casing, annulus, injection wells, and plumbing, for example.

“Repair sealant”, as referred herein, refers to iron or other suitable sealant that may include “silica gel” or other appropriate material that is utilized to fill or cover voids due to corrosion.

“Hydrocarbons”, as referred herein, refer to any number of carbon and hydrogen-containing compounds and/or mixtures of compounds. Illustrative, non-exclusive examples of hydrocarbons according to the present disclosure may include petroleum, oil, crude oil, natural gas, tar, bitumen, and/or mixtures of these materials, as well as any other naturally occurring organic compound that may be distilled or processed from source material, such as subsurface geologic formations or other suitable sources. The terms oil, crude oil, petroleum, and liquid hydrocarbon may all be used interchangeably herein.

“Biologic materials”, as referred herein, include biofilms and planktonic biological materials. The biologic materials also include extracellular polysaccharide, proteins, microbes, and sessile organisms, and planktonic organisms.

“Corrosive environment”, as referred herein, includes a region, area, volume, or a section or portion thereof, or a component or material in such a region, area, volume, section, or portion having (a) detectable levels of any or all of hydrogen sulfide (H₂S) or carbon dioxide (CO₂), bicarbonates, acetic acid, or the like, (b) a non-neutral pH, (c) elevated chlorides and/or temperatures, and any combination thereof.

A “probe”, as referred herein, is a molecular agent that is used for detecting and/or treating target molecules. The probe may include certain probes that are micron-scale, nano-scale, or larger depending on the conduit conditions, flow rate, and best mode of application. For example, the mechanism or type of attachment as well as the method of delivery (e.g., pig-based, sedimentation, etc.) may influence how the probe may be configured or prepared for optimization of evaluation and treatment.

A “nanoprobe”, as referred herein, is a molecular agent that is used for detecting target molecules. The nanoprobe may include certain probes that are nano-scale. For example, the nanoprobes may include functionalized quantum dots, graphene quantum dots, mesoporous silica nanoparticles and/or core/shell structures.

A “tag”, as referred herein, is a component of a probe (e.g., a component of a nanoprobe) that associates with the target. In some embodiments herein, the tag may be DNA, RNA, biofilm tracer or a hydrocarbon tracer. The biofilm tracer may associate with a biofilm component (e.g., extracellular polymeric substance (EPS), proteins, etc.).

“Associate”, as referred herein, refers to any interaction between the tag and the target. For example, the association may include an interaction (e.g., reaction, bond, linkage, and/or electrostatic) between a tag and a target that causes a signal to be generated by the signal generator. Examples of such interactions typically comprise, but are not limited to, electronic, chemical, physical and/or steric interactions between the tag and the target, such as complementary base pair binding between a DNA or RNA primer and the target DNA or RNA, or a chemical reaction between a hydrocarbon and a hydrocarbon tracer.

“Signal”, as used herein, relates to any type of indicator or response (e.g., physical, chemical, electrical, and/or optical) used to indicate the presence of a specific material or environment.

“Reagent”, as used herein, refers to a formulation which allows the probe (e.g., nanoprobe) to be more effectively delivered to the biological, conduit, segment, environment and/or chemical components in the produced fluids (e.g., fluids or hydrocarbons) to be tested.

“Chemical treatments”, as used herein, refers to biocides and/or other types of microbial or non-microbial corrosion inhibitors. Unless the context prohibits otherwise, the use of the term “biocides” herein is intended to include all types of chemical treatments.

As noted above, extensive networks of conduits (e.g., pipes, which may be referred to as pipelines, tubulars, tubing, plumbing herein) carry fluids, such as liquids (e.g., oil and water) and produced gases for petroleum processing and delivery, water treatment, etc. Corrosion causes some of these conduits to become weak or fail, impacting supply and delivery of materials. The extent and distribution of corrosion along conduits is controlled by abiotic and biologic processes. Consortia of multiple organisms may increase the speed of the corrosion processes. The anaerobe-formed biofilms may even promote pit formation and expansion along pipe surfaces. Microbial metabolism promotes corrosion by: (i) changing fluid properties (e.g., pH, alkalinity) and enhancing damage via abiotic processes; and (ii) establishing biofilms (e.g., sulfate reducing bacteria) changes localized conditions and enhances pitting. Further, biofilms decrease efficacy of pigging due to re-establishment of biofilms and resumption of metabolism post mechanical disruption.

Conventional treatments to reduce biofilms involve injecting chemical treatments, injecting chemical treatments, and using mechanical pigs, or other techniques to inhibit or remove biofilm growth (e.g., morphology, multi-organism or metabolism.) The morphology of established biofilms usually responds to flow conditions, but the linkage to why biofilms form where they do initially is more complex. If initiation is promoted by siderophores (e.g., iron-loving microbes), the type of steel used (e.g., distribution of iron) and chemical treatment strategy may to be adjusted address these factors. For example, pipelines used in oil sands industry (e.g., hydrotransport) involve frequent rotation and replacement of conduits because mechanical surface defects are exacerbated by erosion, both of which may be advanced concomitantly. Further, the differences in hydrocarbon quality are another factor that influences the development of different biofilms, which should also be considered in the treatment process. Production conditions include sulfate reducing bacteria, hydrocarbon composition, water chemistry, abundance of sulfate. Accordingly, to enhance conduit integrity and stability, these factors may be utilized to determine what biofilms exist, how the biofilms respond to different hydrocarbons (e.g., from different geologic sources and compositions) flowing through the conduits, and the efficacy of chemical treatment treatments over time.

In one embodiment, the present techniques involve the use of probes (e.g., nanoparticle-molecular probes) for rapidly assessing controls on biofilm development, and mitigating their effects in conduits. The use of probes may involve identifying functional (e.g., DNA (deoxyribonucleic acid)) or phylogenetic (e.g., RNA (ribonucleic acid)) molecular primers associated with microbial communities capable of anaerobic degradation of hydrocarbons in conduits. The probes may include a nanoparticle indicator attached to molecular primers, which may include, but is not limited to, quantum dots, nanoparticle composites, carbon nanotubes or other nanoparticles. These nanoparticle indicators may emit or transmit fluorescent, infrared or visible light, vibration or audible signals, or have other characteristics that provide a mechanism for detection in samples. The system may be nanometer, micrometer, or larger in size. By attaching molecular primers to nanoparticle indicators, the system may provide real-time methods for identifying and treating, areas of active corrosion and/or pit initiation. A comparison of conduit surface integrity with different petroleum products may further target biocide applications to limit additional damage. Probe systems (e.g., nanoparticle system) may need to account for different hydrocarbon compositions, production conditions, mode of transport, microbial populations, and fluid conditions within the pipe or conduit. Further, signal generation may rely more on optical methods (e.g., fluorescence, etc.) with activation being initiated upon association with the biofilm via direct or indirect method.

The present techniques may include various elements to address the biologic materials within the conduits. For example, the first element may include identification of the distribution of biologic materials using the probes (e.g., nanoparticle-molecular probe, which may be referred to as a probe, nanoprobe or nanoparticle probe). The identification may include identifying where are the biofilms within the conduit, identifying what kind of biologic materials are present within the conduit, and determining how the biologic materials respond after biocide treatment(s). This information may be used to determine why the biofilms are growing or developing at the identified locations within the conduit. For example, information may include fluid chemistry, metabolic conditions (e.g., nutrients), change in flow conditions (e.g., bend in conduits), or protected locations (e.g., beneath sanding). Then, the biocides may be delivered directly to the biologic material using the probe, which may include molecular primers, nanoparticle indicators, and biocide or corrosion inhibitor encapsulation beads. This delivery may include dosage similar to current methods or more specifically via encapsulation. For example, the encapsulation may include encapsulating only the biocide component and/or using a delivery system where the system is encapsulated.

Further, the present techniques may include numerous variations to the probes. For example, the probes may include using silicon-based, iron-based, or other mesoporous or nanoparticles types depending on conduit composition, with attached biocide particles to limit additional growth of biofilm and to initiate formation of patches including silicon, iron oxide or iron oxyhydroxide, or the like to temporarily mitigate conduit damage. Sites where biofilms are removed and corrosion has occurred are indicated by pitting and surface roughness. These features cause disruption of localized flow regimes, increased surface area, and enhanced exposure of reactive sites removed and become prime locations for microbial re-colonization. The potential for subsequent multiplex treatment with biocide and pit fillers may result in mitigating the ongoing problem and providing in-situ conduit repair.

The present techniques may include determining the characteristic of the biologic material. For example, the present techniques may identify where and what kind of biologic materials exist along and within the conduit (e.g., located at curves and/or reaches; identify where are they the most prevalent and under what conditions; and identify what is the attachment style and mechanism for biofilms). To evaluate these aspects and account for the various biofilm architectures and microbial consortia, the probes may involve different attachment or association mechanisms. The attachment style and mechanism may include direct attachment and/or indirect attachment. The direct attachment mechanism strategy (e.g., probe-specific tag) relies on a specific association between the microbial DNA/RNA (even if it occurs external to organisms, but is still specific to bacteria or Archaea; or attaches to some compound or property within cell wall). This strategy may be limited because cells trapped or occluded in protective biofilm could be more difficult to access and chemical gradients within the biofilms may alter the properties of the attachment sites. Indirect attachment strategy involves association with biofilm through extra-cellular polymer substances, polysaccharides, enzymes, exudates, or metabolic derivative rather than to the organism DNA/RNA. Although indirect strategies may not be organism-specific (tags), its efficacy in reducing or removing biofilms may still accomplish the ultimate objective. Multifunctional treatments that utilize both direct and indirect methods specifically targeting microbial consortia may prove more effective for biofilm degradation and removal. For example, nanoparticles may also include a barcode or identifiers (e.g., DNA sequence that is not typically found in conduits, reservoirs, etc.). These barcodes may be used to detect the extent of treatment from point to point if flow duration exceeds fluorescence or particle property lifespan.

In the present techniques, the identification and/or treatment may involve a signal generator and/or encapsulated biocidechemical. The properties (e.g., nanotechnology properties) and morphology may be optimized through sedimentation, phase compatibility, activation, stability in flow and deposition to promote efficacy (e.g., density, particle size). The coatings or encapsulation of nanoparticle probe-tag (e.g., DNA, RNA, etc.) and nanoparticles coatings or other properties (e.g., larger encapsulated composite that releases nanoparticle probe biocide treatment upon entering aqueous part of flow) that increase access into and through the biofilm.

The treatment may be configured to deliver biocide during flow where biofilms have established to kill organisms in the biofilm and/or decrease film integrity (e.g., film disruptor such as surfactants); and/or to inhibit attachment and formation of films prior to field installation through pipe coatings (e.g., designed for the biofilms predicted or determined to be present in the conduit). Identifying biofilm type and distribution based on organisms present and/or presence of a diagnostic signature (e.g., biomolecular tag, compound of interest such as proteins, polysaccharides, or extracellular polymeric substances). The present techniques may also include targeting treatment of biologic material either through application of biocides, chemically breaking up biofilm and/or inhibiting additional growth of biofilm, or by “turning off” (e.g., suppressing) the biological signature that promotes sessile living styles. One additional consideration is treating conduits prior to installation with nano-encapsulated biocide that targets likely microbial induced corrosion formers, deters sessile living behavior, or shields sites from abiotic corrosion. These coatings could stimulate the formation of the protective layer that typically forms within conduits after their installation. The application of pipe additives that stimulate the formation of protective layer depends on whether the treatment is appropriate for the particular pipe configuration being employed. Upon replacement of conduits with extensive corrosion, specially treated conduit containing targeted biocide functional coatings could be used. The specially treated conduits or coatings could inhibit initiation of abiotic or microbially induced corrosion, and provide a traceable signature indicating that the coating has been “activated” or released by corrosive processes.

Further, the present techniques may include addition of paramagnetic or other iron-based nanoparticle that may be used to fill pits (e.g., heal pits) or limit additional pitting where biofilms have been removed or are being treated with biocide. Moreover, the present techniques may be used to predict or determine a conduit coating to pre-treat conduits and lessen the likelihood for biofilm initiation and establishment. Further still, the probe (e.g., nanoprobe) may use silicon-based, iron-based or other composition of nanoparticles to limit additional growth of biofilm and to initiate formation of “patches” (e.g., iron oxyhydroxides) to temporarily mitigating conduit damage.

Embodiments herein relate to identifying and treating biologic material (e.g., biofilms, also known as sessile, and planktonic biological material) using probes, such as nanoprobes. In particular, embodiments herein relate to probes useful for detecting biologic materials within conduits and treating such biologic materials. For example, probes (e.g., nanoprobes) may be used to identify the distribution of biologic material (e.g., biofilms); to identify where, what kind, and why biofilms build-up at certain locations and how biofilms respond after biocide treatment. Further, the probes (e.g., nanoprobes) may be configured to provide the biocide directly to biofilm using tags (e.g., molecular primers) attached to nanoparticles with encapsulation characteristics or add an encapsulation bead to the probe.

In some embodiments, the present techniques relate to a method of identifying and treating biological materials of interest may include the use of probes, such as nanoprobes. For example, the present disclosure relates to a probe composition comprising one or more probes, wherein the probe comprises (a) at least one tag capable of associating with a target biological material found in conduits; and (b) at least one signal generator capable of generating a signal when the tag associates with the target biological material. In addition, the method may include: (i) providing a probe composition comprising one or more probes; wherein the probe comprises (a) at least one tag; and (b) at least one signal generator; (ii) introducing the probe composition to a biologic material; and (iii) detecting the presence of a signal generated by the signal generator on association of the tag with a biologic material. The probe may also include a biocide, which is encapsulated, for certain applications, as well. Further, the method may include providing a probe composition comprising one or more probes; wherein the probe comprises (a) at least one tag; and (b) at least one encapsulated biocide; (ii) introducing the probe composition to a biologic material; and (iii) releasing the biocide on association of the tag with a biologic material.

In further embodiments, the present disclosure relates to a method of evaluating a biological material comprising (a) providing a first probe (e.g., nanoprobe); wherein the first probe comprises (i) one or more first tags that associate with a first target biologic material; and (ii) one or more first signal generators; (b) providing a second probe (e.g., nanoprobe); wherein the second probe comprises (i) one or more second tags that associate with a second biologic material (e.g., a different biofilm); and (ii) one or more second signal generators; (c) introducing a probe (e.g., nanoprobe) composition comprising the first probe and the second probe to the biological materials; (d) measuring a first signal; wherein the first signal is generated upon the association of the first probe with the first target biologic material; (e) measuring a second signal; wherein the second signal is generated upon the association of the second probe with a second target biologic material; (f) comparing the first signal to the second signal; and (g) deriving an estimation of the respective distribution and composition of biologic material from contrasting signals. Also, the first and second probes may have a specific biocide, which is encapsulated (e.g., first biocide and second biocide), which are each configured for the respective target biologic material. For example, the method may include the first probe having one or more first tags that associate with a first target biologic material; a first encapsulated biocide may or may not include one or more first signal generators. The first biocide may be released upon association of the first tag with a first target biologic material. Further, the method may include the second probe having one or more second tags that associate with a second target biologic material; a second encapsulated biocide and may or may not include one or more second signal generators. The second biocide may be released upon association of the second tag with a second target biologic material. The distribution and composition may be based on planktonic and/or sessile within the conduit (e.g., nanoprobes activated in response to associations with the targets, but remain in the fluid phase within the conduit and are sampled along the pipe reach).

In yet other embodiments, the present disclosure relates to a system for the characterization of biological materials comprising (a) a probe (e.g., nanoprobe) composition comprising one or more probes (e.g., nanoprobes); wherein the probe comprises (i) at least one tag; and (ii) at least one signal generator; and (b) at least one detector capable of detecting a signal generated by the signal generator.

In other embodiments, the present techniques may be utilized in various configurations. For example, the present techniques may be used in conduits that provide a flow path between a reservoir and a production facility or other fluid management equipment. The present techniques may also be used for applications; such as re-injecting fluids into a reservoir for pressure/production support (e.g., drive). Also, treatment may be added to the fluids to mitigate transfer of organisms from reservoir prior to being entrained in production conduits. In particular, such treatments may lessen H₂S production and decrease the likelihood of H₂S cracking of conduits. In addition, abiotic processes, such as the formation of cathodic (or anodic) sites within the conduit, may stimulate pitting and surface defects that are then advantageous sites for organisms to create biofilms.

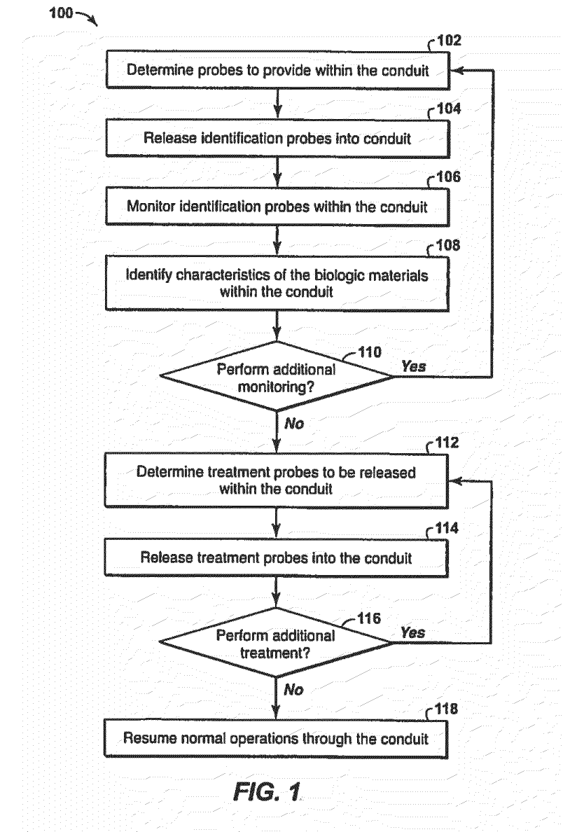

Further, for injection downhole within a well, conventional techniques use nitrate in reservoirs to feed organisms that outcompete the utilization of sulfate resulting in a byproduct increasing H₂S concentrations (reservoir souring). If treatment through this method is stopped, then the sulfate reducing bacteria proliferate due to the treatment not directly targeting the culprits (sulfate reducing bacteria) for reservoir souring. Accordingly, the present techniques may be utilized to manage the biologic material within the injection fluids, the reservoir, and the production fluids. Various aspects of the present techniques are described further in FIGS. 1 to 7.

FIG. 1 is a flow chart 100 for identifying and treating biologic materials in accordance with an exemplary embodiment of the present techniques. In this flow chart 100, probes (e.g., nanoprobes) may be used in an identification stage, as shown in blocks 102 to 110, and a treatment stage, as shown in blocks 112 to 118.

In the identification stage, the biologic materials are identified. At block 102, a determination is made as to what probes (e.g., nanoprobes) are to be provided within the conduit. This determination may be based on a review of prior information about biologic material found within the conduit, or tags that are present. For example, archaea or bacteria contain indicators of life such that a specific sequence of probes can be utilized to identify different biologic material and/or a set of probes having different indicators can be used in sequence and/or parallel. Lab-based experiments using pipe or corrosion coupons may be used to evaluate specificity of attachment or association between the probes (e.g., nanoparticle probes) and the pipe materials. For example, in the case of biological material detection, this determination may include selecting nanoprobes that interact with microorganisms linked to pipe corrosion. At block 104, these identification probes (e.g., nanoprobes) are released into the conduit (e.g., via fluid flow or via a mechanical device, such as a pig). The release of the identification probes may include forming an initial composition by (i) mixing the probes (e.g., nanoprobes) with conduit fluids (e.g., hydrocarbons or water) and/or (ii) mixing the identification probes with a carrier medium (e.g., a carrier fluid and/or solid) and disposing the identification nanoprobes with a carrier medium (e.g., a carrier fluid and/or solid) into the conduit. The identification probes may each include at least one tag and at least one signal generator. The tag may be configured to interact with a specific biologic material, while the signal generator may generate a signal on association of the tag with the specific biologic material. At block 106, the identification probes (e.g., nanoprobes) may be monitored. The monitoring may include monitoring the output of the conduit for changes in the properties of the composition (via visual or UV analyses for nanoparticle probes, similar to breakthrough curves, or via mechanical device with spectroscopic detectors and/or other suitable detection means). For example, the monitoring may include comparing the composition of probes (e.g., nanoprobes) and conduit fluid at the monitoring location with the composition of probes (e.g., nanoprobes) and conduit fluid at the initial release point. One exemplary monitoring method includes evaluating the difference in fluorescence between injection locations and sampling locations. For example, particles may fluoresce initially and then change in response to dilution and retention in the conduit. A change in fluorescence properties or “activation” could be developed to indicate release of biocide and association with organisms. A second exemplary monitoring method includes identifying which particles have been activated (e.g., multiple tag scenario) or retained within the conduit. A third exemplary monitoring method may also include within conduit evaluation, such as with deployment of a smart pig platform. Then, at block 108, the characteristics of the biologic material within the conduit are identified. The identification may include analyzing the changes in composition to determine where the biologic materials are located within the conduit and/or identifying what kind of biologic materials are present within the conduit (e.g., more specific to particular organisms or biofilm properties). Then, at block 110, a determination is made whether to perform additional monitoring in block 102. The additional monitoring may include providing another nanoprobe into the conduit. The additional monitoring may involve evaluating biofilm distribution following traceable pigging run to determine how and where the mechanical treatment was most effective. If no additional monitoring is to be performed, then the treatment stage may begin in block 112.

In the treatment stage, the biologic materials are treated. In block 112, treatment probes (e.g., nanoprobes) may be determined to be released within the conduit. This determination may be based on the identification stage and/or based on prior information regarding biologic material. The treatment probes may each include at least one tag and at least one biocide, which may be an encapsulated biocide. Similar to the identification probe, the tag may be configured to interact with a specific biologic material, while the biocide is activated (e.g., released or engaged) on association of the tag with the specific biologic material. Also, the treatment probe may include a signal generator to generate a signal on association of the tag with the specific biologic material (which may indicate that the target was treated with biocide). Then, at block 114, the treatment probe (e.g., nanoprobe) may be released within the conduit. The release of the treatment probes may include forming an initial composition by (i) mixing the treatment probes (e.g., nanoprobes) with conduit fluids (e.g., water and hydrocarbons within the conduit) and/or (ii) mixing the treatment probes with a carrier medium (e.g., a carrier fluid and/or solid) and disposing the treatment probes with the carrier medium (e.g., a carrier fluid and/or solid) into the conduit. Further, the release may involve using a mechanical device, such as a pig, to release the treatment based on positive nanoparticle identification indicators. At block 116, a determination is made whether an additional treatment should be performed. This determination may include analyzing the changes in composition, morphology, chemistry or distribution of biofilms to determine how the biocide is interacting with the biologic materials (monitoring for signals or other indicators) and/or performing the identification stage to determine if the biologic material is present. For example, with multiplex treatments, it may be advantageous to determine which is working or is more effective for the treatment step. If additional treatments are to be performed, the process may determine the treatment probes in block 112. However, if no additional treatment is to be performed, then the normal operations may continue in block 118. The normal operations may involve continuing to provide fluids through the conduit (e.g., multiphase fluid flow, water, etc.). These applications may include conduits conveying hydrocarbons, waste treatment effluent, water circulation, and/or heating/cooling materials. Further, frequent and/or scheduled monitoring and treatment may constrain biofilm growth rates, how likely the biofilms are to re-establish at particular locations after treatment, biocide dosing and efficacy evaluations, and therefore enhancing long-term corrosion forecasting based on targeted treatments and evaluation (e.g., the process may be repeated to monitor the temporal aspects of abiotic and biologically influenced corrosion). For example, temporal evolution of pipe integrity and susceptibility to abiotic and biologically influenced corrosion may provide enhanced additional modelling inputs that may provide for changes in the management plan based on increased evaluation options over time, improving predictions of when the pipe should be replaced or provide enhanced predictions for the overall lifespan of the pipe materials.

Beneficially, the present techniques may use probes, which may be nanoprobes, to identify the location of biologic materials within conduits and for the treatment of biologic materials within conduits. The probes may be used to detect particular biologic materials, biofilm components, particular microorganism DNA and/or RNA, and/or metabolic products of the microorganisms. The biofilm components may include extracellular polymeric substances, polysaccharides, proteins, and/or lipids.

Alternatively, the present techniques may be used to treat corrosion locations within the conduit. The method may involve identifying and treating corrosive environments of interest within a conduit. The method may include similar blocks to those noted above in FIG. 1. However, the tag may be associated with corrosive environments instead of biologic materials. The corrosive environment may include corrosion by-products, cathode/anode reaction locations, and/or redox potential or locations. These corrosive environments may be disposed under sediments or sludge, for example. The corrosion inhibitor may be a chemical compound that, when added to a liquid or gas, decreases corrosion of the material.

As an example, the method may include providing a probe composition comprising one or more probes (e.g., nanoprobes); wherein each of the one or more probes comprises: a tag; and one or more of a signal generator (e.g., nanoparticle or inorganic fluorophore) and a corrosion inhibitor, wherein the probe is configured to generate a signal when the tag associates with a target corrosive environment, if the signal generator is present in the probe, and release the corrosion inhibitor when the tag associates with the target corrosive environment, if the corrosion inhibitor is present in the probe; releasing the probes into a conduit; if the probe composition includes one or more probes having the signal generator, detecting the presence of a signal generated by the signal generator on association of the tag with the target corrosive environment. The tag may be an electrochemical tracer, redox sensitive tracer, reactive functional tracer, anode potential tracer, cathode potential tracer and/or the like. The particle may include a silicon nanoparticle, a mesoporous silica nanoparticle, a graphene quantum dot, a core/shell composite, a cadmium selenide nanoparticle, a cadmium-sulfide nanoparticle, a quantum dot, a nanoparticle composite, a nanocrystal, and a carbon nanotube.

As noted above, the treatment and identification probes (e.g., nanoprobes) may be utilized to detect a target biological material and/or to treat the target biological material. These probes include a tag along with a biocide and/or signal generator, which depends upon the specific configuration. The tag is a component of a probe that associates with the target biologic material. The tag may be a DNA, a RNA, a biofilm component tracer, or a cathode/anode site-specific tracer. As noted above, the association between a tag and a target biologic material is any interaction (e.g., electronic, chemical, physical, and/or steric interactions) between the tag and the target biologic material that causes a signal to be generated by the signal generator. The interactions may include complementary base pair binding between a DNA or RNA primer and the target DNA or RNA, or a chemical reaction between a biofilm and biofilm component tracer. The tag may be biomolecular or geomolecular, cathode/anode sites-specific tags, which are discussed further below.

Biomolecular tags typically associate with biological material targets, such as microorganisms, in particular the genetic material, cell wall material, or cell membrane material of microorganisms. Examples of such biomolecular tags include DNA and RNA primers. Such DNA and RNA primers may be complementary to sections of the microorganism genetic material characteristic of a particular species; and/or sections of the genetic material that encode for a specific metabolic function, such as metabolizing hydrocarbons (e.g. aqueous phase hydrocarbons); or sections of the genetic material that encode for processes making use of the products of this metabolysis, such as sulfate reduction, acid production, methanogenesis, and the like. Biomolecular tags may be identified and designed by any means known in the art, such as pyrosequencing-based metagenomics, single cell genomics, or other well-known techniques. Biomolecular tags may also be purchased from commercial sources.

Geomolecular tags typically associate with particular hydrocarbons, for example, a hydrocarbon tracer compound that associates with compounds of sludge or other deposits within the conduit. The sludge components may include hydrocarbons, salt, water, microbes, chlorides, organic acids, and corrosion byproducts. Sludge properties and associated corrosion varies as a function of hydrocarbons and produced water composition. For example, crude oils with organic acids and relatively more water soluble compounds and asphaltenes, typically have higher microbial populations that are known to induce corrosion. Waxy oils tend to coat conduit surfaces, which limits water availability for microbial growth. As such, the geomolecular tags may be utilized to differentiate and distinguish the sludge composition and corrosion types.

In some embodiments, the geomolecular tags may comprise a functional group that attaches to a functional group of interest in the target. In other embodiments, the geomolecular tag may react with the target hydrocarbons. In yet other embodiments, the geomolecular tag may sorb onto the target hydrocarbons. “Sorbing” or “sorption” includes adsorption, chemical adsorption (i.e., chemisorption), absorption, and/or physical adsorption (i.e., physisorption). In other embodiments, the geomolecular tag may associate with the hydrocarbons by partitioning in the presence of hydrocarbons. “Partitioning” means the relative solubility of a compound in a mixture of two or more immiscible solvents. For example, a compound may partition at the interface of a mixture of oil and water. Where the compound is hydrophilic, the compound may be preferentially found in the water layer and is referred to as having a low partition coefficient. Where the compound is hydrophobic, the compound preferentially migrates to the oil layer and is referred to as having a high partition coefficient. Amphiphilic compounds may partition at the interface of the oil/water mixture. It may be preferential to use encapsulation coatings with different partitioning characteristics to reach the target of interest (e.g., under deposit corrosion or exposed biofilms).

In some embodiments, the probes may include different tags. For example, the probe may have two or more tags or coatings, alternately three or more tags, or the like. Further, the tag may be biomolecular or geomolecular to associate with a target biologic material. “Target biologic material” means a target biologic material of interest with which the tag associates. In some embodiments herein, the target is at least one of hydrocarbons, microorganisms that metabolize hydrocarbons, and compounds produced by the microorganisms, such as metabolic products or corrosion byproducts. In other embodiments, the target is one of genetic material of microorganisms that metabolize the chemical components in the produced fluids or geological material within produced fluids, polysaccharides found on the cell walls of microorganisms that metabolize the chemical components in the produced fluids or geological material within produced fluids, and proteins, lipids or sterols found in the cell membranes or within biofilms of microorganisms that metabolize the chemical components in the produced fluids or geological material within produced fluids. Examples of targets include hydrocarbons such as toluene, benzene, ethyl benzene, and xylene, genetic material of microorganisms, for example Alcanivorax spp., and/or metabolic byproducts such as 2-methylbenzyl succinate for toluene (See, e.g., Young and Phelps, 2005, Metabolic biomarkers for monitoring in situ anaerobic hydrocarbon degradation).

For identification probes and certain treatment probes, the probe may also include a signal generator. The signal generator refers to a molecule that generates a signal when the tag associates with the target environment, be it biologic or corrosive in nature. The signal generator may be a nanoparticle, which is particle with at least one dimension less than 100 nm or other suitable scale. Some examples of nanoparticles include nanopowders, nanoclusters, and nanocrystals. These nanoparticles may be used in sensory applications, as they have size-dependent properties. That is, a bulk material typically has constant physical properties regardless of its size, but nanoparticles may vary physical properties. Size-dependent properties may include quantum confinement in semiconductor particles, surface plasmon resonance in some metal particles and superparamagnetism in magnetic materials. The properties of materials change as their size approaches the nanoscale and as the percentage of atoms at the surface of a material becomes significant. For bulk materials larger than one micrometer the percentage of atoms at the surface is minuscule relative to the total number of atoms of the material. Accordingly, nanoparticles are useful as signal generators. The signal generator may have a specific lifespan to lessen any potential negative effects on the conduit and/or fluid properties.

For example, the signal generator may include inorganic signal generators, such as inorganic fluorophore, silicon nanoparticle and/or a cadmium selenide nanoparticle. Unlike conventional tracing and imaging technologies (e.g., organic dyes as markers or probes) that are susceptible to degradation by photoexcitation, room light, or high temperatures, inorganic signal generators may be used in conduit environments (e.g., pressures and/or temperatures). Multiple shell nanoparticles may be used to enhance signal intensity and diverse fluid types. For example, some nanoparticles may preferentially have photoexcitation, particularly for those samples taken from the conduit (fluids) and analyzed in lab settings. Additionally, inorganic signal generators are not likely to stick to non-target surfaces and/or associate indiscriminately with sediments, as compared with organic tracers unless functionalized to perform specific associations.

The nanoparticle may also include one or more of a the particle is one or more of a silicon nanoparticle, a mesoporous silica nanoparticle, a graphene quantum dot, a core/shell composite, a cadmium selenide nanoparticle, a cadmium-sulfide nanoparticle, a quantum dot, a nanoparticle composite, a nanocrystal, and a carbon nanotube. “Quantum dots” are nanometer sized semiconductor materials typically made from semiconductor elements such as silicon or germanium, or semiconductor compounds, such as CdS or CdSe. These nanoparticles may differ in color depending on their size. Quantum dots may be used as signal generators based on the unique properties, such as electrical and nonlinear optical properties. Quantum dots may also emit light if excited, with the smaller the dot, the higher the energy of the emitted light. Advantageously, quantum dots do not degrade rapidly and may not stick to other materials found in core samples or in the wellbore.

The signal provided by the signal generator may include a variety of indications of a specific biologic material or environment of interest. The signal may include one or more of an audible notification, a sonar notification, an acoustic notification, a visible notification, a radiation notification, an infrared notification, electrical notification, and a fluorescent notification. As a result, the signal may be a change of color or fluorescence or the intensity of a signal may change on association of the target of interest (such as a biologic material) interest with the tag. For example, silicon nanoparticles take on different colors depending on the size of the nanoparticle. Accordingly, the probe may have a different color or intensity of color than the tag specific target molecule upon activation. Further, the signal is generated on association of the tag with the target biologic or corrosive environment. The association of the tag with this target comprises one or more of sorbing, partitioning, ionic bonding, hydrogen bonding, adsorption, covalent bonding, adhesion, electrostatic interactions. If a suitable complementary target of interest is absent, no signal is generated or signal fluorescence decreases, such as due to association and retention of nanoparticles by biofilm or due to association with planktonic organisms. Accordingly, the absence of a signal indicates that the target material is not present. If a suitable complementary target is present, a signal is provided, which indicates the presence of the target environment.

In some embodiments, the signal generators may be configured to not produce a signal until activated. For example, the signal generators may be activated by an ultraviolet (UV) light or a radio signal, while others may automatically activate to produce the signal. For those types of signal generators, an activation process may be used. In some applications, activation can take the form of detaching an agent that prevents the signal generator from emitting a signal, or quenches the signal emitted from the nanoprobe.

Based on the type of signal notification, the detection methods may vary to accommodate the different signals. The change in a signal, presence of a signal, or absence of a signal is preferably detectable by some means known in the art, such as using one or more of a UV-Vis (Ultraviolet-Visible) spectrometer, IR (infrared) spectrometer, a fluorimeter, a Raman spectrometer, and/or a sonar detector. Alternatively, the signal may be detected visually or audibly by an observer or monitoring device. For example, a signal generator that provides a visible color change as the notification may be immediately discernable by a visual observation or by a spectrometer, while a signal generator that provides a fluorescence may be detectable by standard fluorometric techniques, such as by using a fluorimeter. Accordingly, the detectors for the signal generators may be analyzed in any convenient manner. Some such signals may include binary indication, for example, because they indicate that a particular biologic material either is or is not present in the conduit. Other detectors may analyze the presence of the signal and its magnitude and/or intensity to provide different levels of presence. For example, the presence of the signal may indicate that a particular biologic material is present in the conduit and the intensity of the same or another signal may provide an indication of approximately how much of the particular biologic material is present. Further still, the detection may include detecting different types of signals to indicate the different types of biologic material. For example, the detection may include a visible color change as the notification of a first biologic or component of interest, which is detected by a spectrometer, and fluorescence as the notification of a second biologic or component of interest, which may be detected by a fluorimeter.

To analyze the detected signals, various techniques that are known in the art may be used for interpretation of the signals. For example a calibration curve may be used to correlate the fluorescence signature or signatures to, as the previous example notes, the biologic material presence, extent and diversity of the microbiology and biological material in the conduit. As may be appreciated, suitable corrections or adjustments may compensate for biasing factors such as, for example, contamination from marine or other organisms living in the sediments and/or from other biologic material not associated with conduit fluids and/or not associated with corrosion.

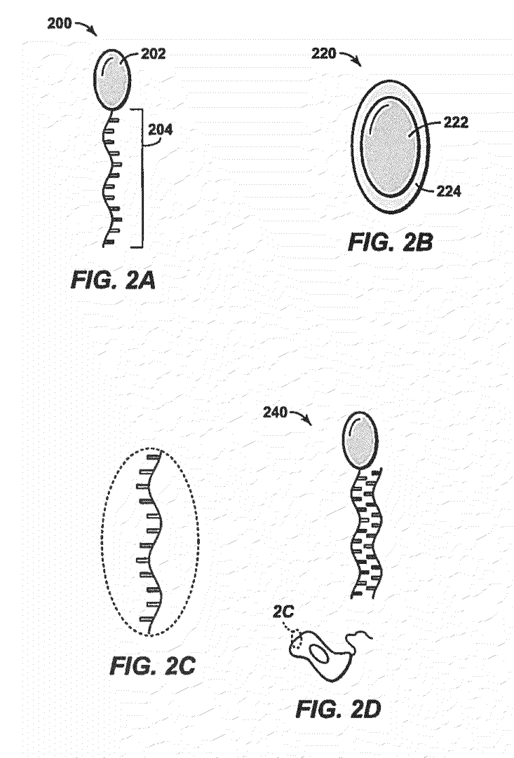

FIGS. 2A, 2B and 2C are diagrams of identification probes 200, 220 and 240 in accordance with an exemplary embodiment of the present techniques. These identification probes 200, 220 and 240, which may be nanoprobes, may be utilized to detect a target biological or corrosive environment and/or to treat the target biological or corrosive environment. The identification probes 200, 220 and 240 have a probe composition that includes at least one tag capable of associating with a target material and at least one signal generator capable of generating a signal when the tag associates with the target material. These probes 200, 220 and 240 may be sub-micron scale in size. That is, depending on the tag and signal generator, the probe may not necessarily be nanometer scale in size.

In FIG. 2A, the probe 200 includes a RNA or DNA primer tag 204 that is attached to a particle indicator 202. In FIG. 2B, the probe 220 includes an EPS (extracellular polymeric substance) specific tag 224 (e.g., coating) that is attached to a particle indicator 222. In FIGS. 2C and 2D, the probe 240 includes an EPS specific tag that is attaching to a biologic material of interest. In an alternative embodiment for geomolecular applications, the tag 224 may be specific to corrosive environments or components of interest present in sludge.

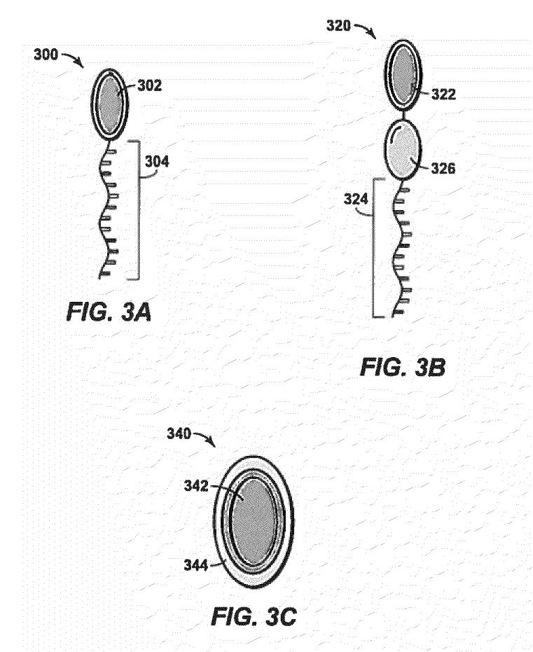

Similar to the identification probes, FIGS. 3A, 3B and 3C are diagrams of treatment probes 300, 320 and 340 in accordance with an exemplary embodiment of the present techniques. These treatment probes 300, 320 and 340, which may be nanoprobes, may be utilized to treat a target biological or corrosive environment. The treatment probes 300 and 340 have probe compositions that include at least one tag capable of associating with a target biologic or corrosive environment and one or more of an encapsulated or corrosion inhibitor capable of being released when the tag associates with the target biologic or corrosive environment. For probe 320, the probe includes a probe composition that include at least one signal generator capable of generating a signal, at least one tag capable of associating with a target material and one or more of an encapsulated biocide capable of being released when the tag associates with the target material. The at least one signal generator is capable of generating a signal when the tag associates with the target material, which may be utilized to indicate the location of the association. These probes 300, 320 and 340 are typically sub-micron scale in size. That is, depending on the tag, the encapsulated biocide and signal generator, if present, the nanoprobe may not necessarily be nanometer scale in size.

In FIG. 3A, the nanoprobe 300 includes a RNA or DNA primer tag 304 that is attached to an encapsulated biocide 302. In FIG. 3B, the probe 320 includes a RNA or DNA primer tag 324 and a particle indicator 326 that is attached to an encapsulated biocide 322. In FIG. 3C, the probe 340 includes a tag 344 (e.g., coating) that is attached to an encapsulated biocide 342. In an alternative embodiment for other applications, the tag 324 or 344 may be specific to corrosive environment or components of interest present in sludge.

To release the probes, a carrier medium may include a reagent. The reagent may comprise a fluid, which promotes pouring, spraying, aerial dispersion, or dissolution into or onto the conduit fluid, which may be selected from the group consisting of hydrocarbons fluids, water, brine, organic solvents, and the like, or a mixture thereof. In other embodiments, the reagent comprises a solid, thereby allowing another mechanism of dosing the probe (e.g., nanoprobe) composition into the conduit fluid; promoting dissolving of the probe in a fluid within the conduit, and/or allowing timed release of the nanoprobe within the conduit. Further, the reagent may be selected to amplify the signal by any means known in the art. Also, the reagent could take the form of, for example, a powder, pellet, solution, or suspension.

Also, the carrier medium may include a mixture of solid and/or fluid to further manage the release of the nanoprobes at certain locations within the conduit. For example, it may be appropriate to encase, protect, or otherwise carry the probe composition in a carrier medium. For instance, dried particles with hydrophilic coating may dissolve once powder or sand-size materials are introduced into the conduit. The selection of the carrier medium may be based on the environment within the conduit in which the probes are to be applied. For example, the potential adverse effects of fluid chemistry, pH, temperature and/or pressure on the probes' durability may prompt the selection of a carrier medium that provides resist to these and other environmental parameters for a certain time. For example, pressure is tough to account for however durable particle systems should be tested to withstand range of conduit conditions, but optimization may be involved for change over time. The configuration may involve treatment to ensure that it does not result in conduit damage, cause scale formation, or impact fluid treatments, such as at separator facilities where pressure and temperature may change drastically. For purposes of this disclosure, such carrier media should be considered as reagents. Also, the probe composition may be part of an article. The article may be in the form of a platform, sheet, film, net, mesh, and/or similar structures.

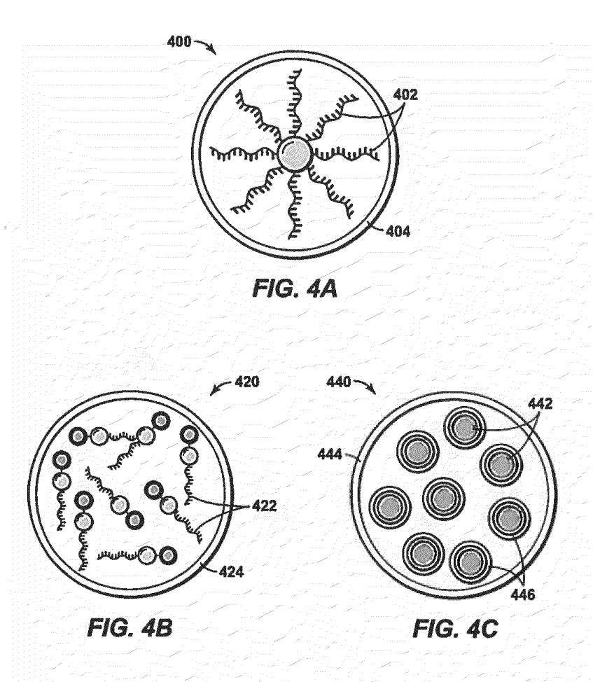

In addition, the identification probes and treatment probes may also be incorporated into a composite or encapsulation that may include certain particle coatings to manage the location that the nanoprobes are released. FIGS. 4A, 4B and 4C are diagrams of encapsulated probe systems 400, 420 and 440 in accordance with an exemplary embodiment of the present techniques. These encapsulated probe systems 400, 420 and 440, which may be nanoprobe systems, may be utilized to manage the release of the probes from the encapsulation within the conduit.

Encapsulation may include coatings that preferentially transfer treatment systems to target locations. Aqueous phase or preferred phase may be used for maximum usage (e.g., salt versus freshwater, and/or attachment to sludge). Encapsulation may include fluid property and time released/dissolvable coatings to ensure that the biocide is delivered in the appropriate phase and in close proximity to the location of interest. Also, encapsulation may comprise multiple layers, which may provide for appropriate phase transfer, an option for time release within the same particle system. Encapsulation could also determine product or system format. For example, the format may include a liquid or fluid portion within the encapsulation sphere. The delivery may result from a trigger event that opens the encapsulation sphere. For example, the trigger event may include certain solution conditions, magnetic properties or other signals that are utilized to activate the release. One mechanism for releasing particles (e.g., nanoparticles) from encapsulation could be upon deployment from a pig.

The probe delivery may include different encapsulation techniques. For example, encapsulation sphere may be configured to release probes based on phase transfer, fluid properties (e.g., gating, such as redox-based gating or sulfide reduction based gating) and/or time release. That is, the release mechanism may include time release, trigger release or the like. Further, it may be preferable to have a platform of nanoparticles, which are released over time as fluid flow passes the platform.

In FIG. 4A, the encapsulated probe system 400 includes various identification probes 402 (e.g., nanoprobes), which each have RNA or DNA primer tag that is attached to a signal generator. These identification probes 402 are encapsulated by an encapsulation layer 404, which may be configured to be a hydrophilic coating, time release coating or other suitable release mechanism. In FIG. 4B, the encapsulated probe system 420 includes treatment probes 422 (e.g., nanoprobes), which each include a RNA or DNA primer tag that is attached to an encapsulated biocide and signal generator. These treatment probes 422 are encapsulated by an encapsulation layer 424, which is configured to be a hydrophilic coating, time release coating or other suitable release mechanism. The hydrophilic encapsulation or coating may be used to release a biocide in the aqueous phase of the conduit fluid (e.g., locations where the biofilms are likely to exist) and limit biocide release in the oil phase. The release of the biocide in the oil phase is a typical problem with current methods. In FIG. 4C, the encapsulated probe system 440 includes biocide 442 and tag or coating 446. The biocides are encapsulated by an encapsulation layer 444, which is configured to be a hydrophilic coating, time release coating or other suitable release mechanism. The encapsulation layers 404, 424 and 444 may also be referred to as encapsulation spheres or other encapsulation volumes. The delivery mechanism may be preferential to have multiple treatment probes with a range of association types within one encapsulation sphere (e.g., biofilm, microbial targets, reactive sites, or cathode/anode locations). Encapsulation may be customized to attach to materials or components expected to be present in a particular material or environment of interest.

As noted above, particle-based sensing and applications utilizes the unique properties of nanoparticles and the flexibility for adding a range of functionalization to initially address targeted treatment of microbially-induced conduit corrosion. One application of these probes, such as nanoprobes, may involve using probes configured to selectively interact with microorganisms linked to pipe corrosion. For example, nanoprobes may include nanoparticles that are functionalized with tags that specifically target microorganisms (e.g., sulfate reducers), byproducts of their metabolism (e.g., alkyl succinates), corrosive by-products, or their biofilm components (e.g., proteins, lipids). These probes may be configured to operate in conduit environments with conduit fluids (e.g., fluid compositions, temperatures, pressures, and flow conditions (e.g., pH, salinity and/or flow rate) for which the nanoprobes may be exposed to during identification and/or treatment stages of operations. Further, these nanoprobes may be configured to for distribution and treat biofilms associated with microbially influenced corrosion. That is, the nanoprobes and/or biocides may be encapsulated to enhance the efficacy of identification and treatment.

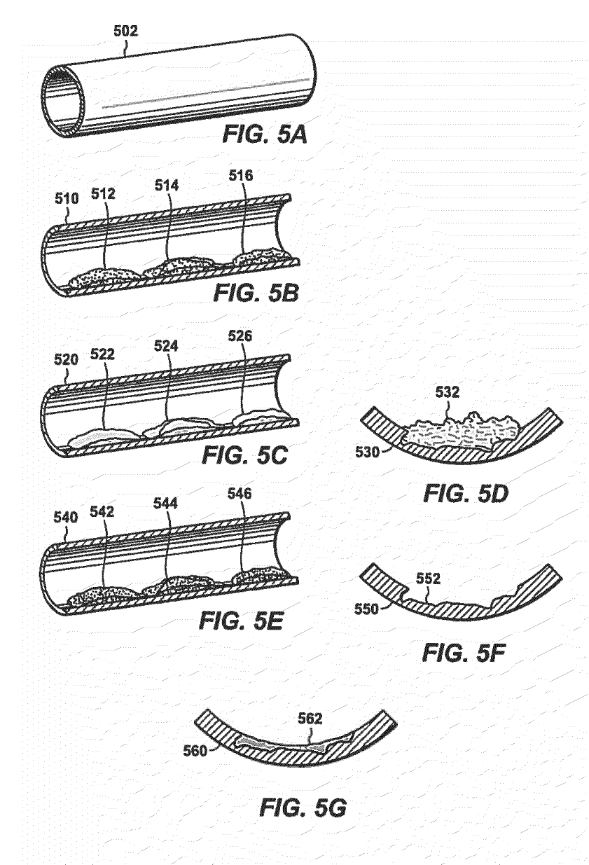

Some embodiments herein relate to the use of probes in pipeline systems, which include a variety of conduits. These probes, which may be nanoprobes, may be used to assess the presence of biologic and corrosive environments. FIGS. 5A-5G are diagrams of the use of the probes for a conduit in accordance with an exemplary embodiment of the present techniques. These diagrams are an example of a pipe application where probes, such as nanoprobes, are used to identify different microorganisms responsible for biofilm growth. In particular, a conduit 502 is provided and used for transporting hydrocarbons (FIG. 5A). After some period of operation, various biologic materials, such as biofilms 512, 514 and 516, develop on the surface of the conduit 510 (FIG. 5B), which is a partial section of the conduit 502. These biofilms 512, 514 and 516 may represent the same or different types of biologic material. Once the probes are released within the conduit, the probes may associate with the biofilms 522, 524 and 526 on the surface of the conduit 520 (FIG. 5C), which represents the partial section of the conduit 502 at a later time from conduit 510. This interaction of the probes and the biological material is shown in the biofilm 532 and conduit 530 (FIG. 5D), which is a cross section of the conduit 520. Once the biocide has been released to the biofilms, the pits 542, 544 and 546 may form in the internal surface of conduit 540 (FIG. 5E), which represents the partial section of the conduit 502 at a later time from conduit 520. This pitting is shown in the pit 552 and conduit 550 (FIG. 5F), which is a cross section of the conduit 540. Then, a protective film 562 may be established in the conduit 560 (FIG. 5G). The protective film 562 may form from particle compositions, such as a repair sealant. The repair sealant may include iron or other suitable sealant that may include “silica gel” or other appropriate particle material that is utilized to fill or cover voids due to corrosion. Custom pipe coatings, such as containing encapsulated biocide, may be determined prior to installation of the conduits.

To monitor the application of the identification and/or treatment probes (e.g., nanoprobes), the process may involve different techniques. For example, the process may release identification probes to determine if the biologic material is present within the conduit. These identification probes may involve one or more probes having different tags that are selective to different biologic materials. In this configuration, one or more detectors may be disposed downstream of the location where the probes are released into the conduit or may be fixed at locations along the conduit to monitor for signals provided by the signal generators.

Alternatively, a monitoring tool may be utilized to travel through the conduit to determine distribution or to analyze the signal generated by the probes. Typically, monitoring for corrosive conditions along the entire length of pipelines is not performed or involves shutting down the pipeline to monitor. Further, corrosion modeling typically lacks quality field data for full pipeline corrosion prediction and the feedback on changes in corrosion mitigation is time delayed in conventional techniques. Accordingly, the monitoring tool may use multi-sensor technology to provide enhanced data analysis. The multi-sensor technology may be used to perform analysis of data to maximize modeling predictions and/or verification of nanoprobe operations. Further, this monitoring tool may be used to collect data on leading corrosion indicators and may be deployed in conduits with minimum disturbance. The monitoring tool may provide data analysis that may be combined with corrosion and flow modeling to track pipeline health, improve/optimize, and determine current and future integrity and operations.

Beneficially, a monitoring tool may enhance integrity monitoring by providing active (e.g., real-time) monitoring and may even be combined with corrosion and flow modeling techniques. Further, the monitoring tool may be used to monitor leading corrosion indicators within the conduit at different stages of operation (e.g., addresses gaps in data and feedback frequency between monitoring and inspection). Also, the monitoring tool may be used to validate models and design assumptions on the conduits and nanoprobes used to maintain the conduit. Moreover, the monitoring tool may use hardware and software platforms that are capable of being implemented in a variety of environments and for a variety of conduit sizes. Further still, the monitoring tool and its use may reduce corrosion associated costs, be used in a variety of pipeline assets; enable proactive monitoring and maintenance operations decision making and lessen pipeline integrity uncertainty. In particular, smart mechanical devices (e.g., smart pigs) may include sensors to be able to identify pitting, and other surface defects. Further, combining smart pigging methods with targeted chemical treatment may result in the maximum benefit being achieved.

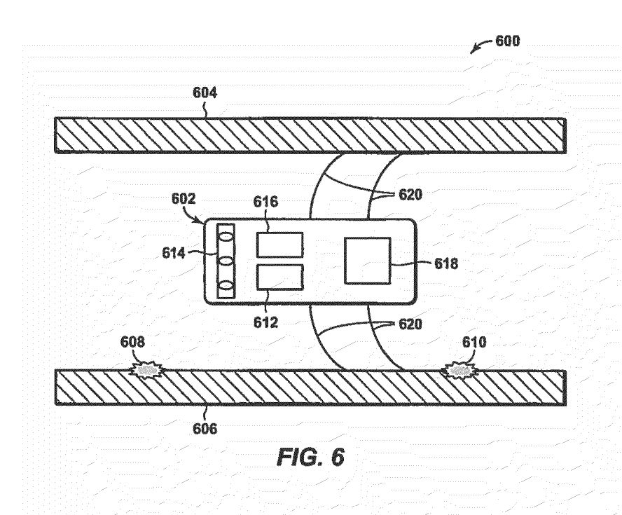

As an example, FIG. 6 is a diagram 600 of a monitoring device or tool 602 (e.g., a smart pig) for use with probes in a conduit in accordance with an exemplary embodiment of the present techniques. The conduit has a first surface 604 and a second surface 606. On the second surface 606, various biofilms 608 and 610 are present. These biofilms 608 and 610 may include microorganisms that can directly or indirectly cause corrosion, such as sulfate reducing bacteria, acid producing bacteria or methane oxidizers.

To monitor the surfaces 604 and 606, the monitoring tool 602 may travel through the conduit to monitor environmental conditions, such as temperature, pressure, water cut, solid deposition, fluid velocity and/or fluid density. The monitoring tool 602 may include control unit 612 that is configured to communicate and operate various spectroscopic analysis, detectors, such as acoustic sensors 614, fluid chemistry module 616 and physical sensors 618 having surface probes 620. The physical sensors 618 may be configured to obtain pitting measurements, obtain surface roughness measurements, etc. The monitoring tool 602 may also include components that provide positioning and location, such as distance and coordinates using gyroscope methods, for example. The monitoring tool 602 may be utilized to repair or treat the conduit if the composition can be deemed appropriate for pipe conditions. The monitoring tool 602 may include a deployment module configured to release probes to a specific location within the conduit.

To provide the monitoring functionality, the monitoring tool may include a control unit (e.g., a computer system or processor) and/or may include a remote control unit (e.g., (e.g., a computer system or processor that is external to the conduit) (not shown) that is in communication with the control unit. The control unit 612 may be configured to manage the multi-sensor technologies that are being measured and to perform analysis of the measured data. The control unit 612 may process this data to provide modeling predictions and/or to verify nanoprobe operations. Further, the control unit 612 may be configured to obtain measurement data and to process the data to provide indications on leading corrosion indicators. The modeling and/or data may be communicated to the remote control unit or other suitable computer system.

As an example, a system or monitoring tool for identification and treatment of a biologic material and/or corrosive environment within a conduit may include various components to enhance the operations. The system may include a delivery device that is configured to store a probe composition having one or more probes. The probes may include a tag capable of associating with a target material; and a signal generator and chemical treatments such as a biocide and/or a corrosion inhibitor. The probe may be configured to generate a signal when the tag associates with a target material, if a signal generator is present in the probe. Also, the probe may release the biocide when the tag associates with the target corrosive environment, if a biocide is present in the probe. In addition, the probe may release the corrosion inhibitor when the tag associates with the target corrosive environment, if the necessary corrosion is present in the probe. The one or more probes may be nanoprobes and/or the probe composition may include a reagent.

Further, the system or monitoring tool may include one or more detectors capable of monitoring the target materials interactions with the one or more probes. The detector may include a monitoring tool that is configured to be disposed within the conduit and measure data within the conduit with one or more sensors. The sensors may be configured to detect a signal generated by the signal generator, which may also include one or more of a ultra-violet-visible wavelength (UV-Vis) spectrometer, IR spectrometer, a fluorimeter, a Raman spectrometer, and/or a sonar detector. Further, the sensors may be configured to detect electrochemical signals (e.g., potentiometric, polarization and/or galvanic responses).

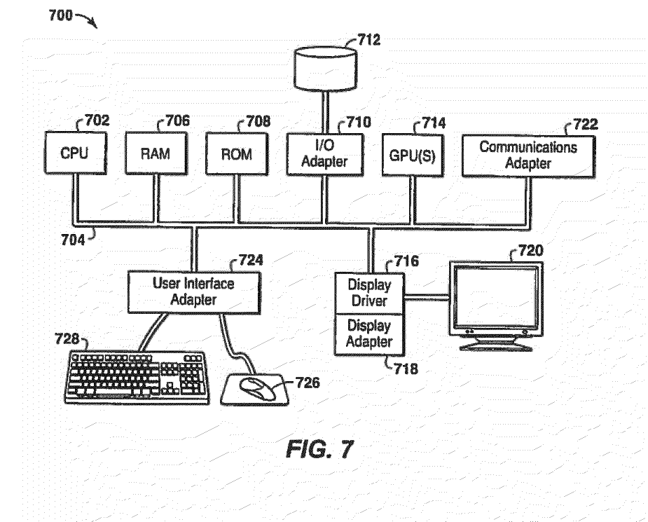

As an example, FIG. 7 is a block diagram of a computer system 700 that may be used to perform any of the methods disclosed herein. In particular, this computer system or portions of this computer system may be used for the control unit 612 and/or remote control unit. A central processing unit (CPU) 702 is coupled to system bus 704. The CPU 702 may be any general-purpose CPU, although other types of architectures of CPU 702 (or other components of computer system 700) may be used as long as CPU 702 (and other components of system 700) supports the inventive operations as described herein. The CPU 702 may execute the various logical instructions according to disclosed aspects and methodologies. For example, the CPU 702 may execute machine-level instructions for performing processing according to aspects and methodologies disclosed herein.

The computer system 700 may also include computer components such as a random access memory (RAM) 706, which may be SRAM, DRAM, SDRAM, or the like. The computer system 700 may also include read-only memory (ROM) 708, which may be PROM, EPROM, EEPROM, or the like. RAM 706 and ROM 708 hold user and system data and programs, as is known in the art. The computer system 700 may also include an input/output (I/O) adapter 710, one or more graphics processor units (GPU) 714, a communications adapter 722, a user interface adapter 724, and a display adapter 718. The I/O adapter 710, the user interface adapter 724, and/or communications adapter 722 may, in certain aspects and techniques, enable a user to interact with computer system 700 to input information.

The I/O adapter 710 preferably connects a storage device(s) 712, such as one or more of hard drive, compact disc (CD) drive, floppy disk drive, tape drive, etc. to computer system 700. The storage device(s) may be used when RAM 706 is insufficient for the memory requirements associated with storing data for operations of embodiments of the present techniques. The data storage of the computer system 700 may be used for storing information and/or other data used or generated as disclosed herein. The communications adapter 722 may couple the computer system 700 to a network (not shown), which may enable information to be input to and/or output from system 700 via the network (for example, a wide-area network, a local-area network, a wireless network, any combination of the foregoing). User interface adapter 724 couples user input devices, such as a keyboard 728, a pointing device 726, and the like, to computer system 700. The display adapter 718 is driven by the CPU 702 to control, through a display driver 716, the display on a display device 720. Information and/or representations of one or more 2D canvases and one or more 3D windows may be displayed, according to disclosed aspects and methodologies.

The architecture of system 700 may be varied as desired. For example, any suitable processor-based device may be used, including without limitation personal computers, laptop computers, computer workstations, and multi-processor servers. Moreover, embodiments may be implemented on application specific integrated circuits (ASICs) or very large scale integrated (VLSI) circuits. In fact, persons of ordinary skill in the art may use any number of suitable structures capable of executing logical operations according to the embodiments.

In one or more embodiments, the method may be implemented in machine-readable logic, set of instructions or code that, when executed, performs a method to determine and/or estimate the seepage locations. The code may be used or executed with a computing system such as computing system 700. The computer system may be utilized to store the set of instructions that are utilized to manage the data, the different measurement techniques, and other aspects of the present techniques.

For example, the present techniques may include a computer system or processed based device that is configured to deliver and/or monitor the probes provided within the conduit. The set of instructions may be configured to detect the presence of a signal generated by the signal generator on association of the tag with the target biologic material. This set of instructions may be configured to detect audible signals, sonar signals, acoustic signals, visible signals, and fluorescent signal. The system may include a set of instructions configured to interact with sensors to communicate and process data from a UV-Vis spectrometer, IR spectrometer, a fluorimeter, a Raman spectrometer, and a sonar detector. Further, if different probes are used (e.g., first probe associated with first biological target and a second probe associated with a second biological target), the set of instructions may be configured to compare the first signal to the second signal; and derive an estimation of the respective proportions of water and target in the chemical components in the produced fluids.

As may be appreciated, the present techniques may include various applications. For example, the present techniques may include verifying nanoprobes, performing corrosion modeling, modeling predictions of the conduit; performing modelling to predict corrosion indicators; integrating corrosion and flow modeling to track pipeline health and operations; validate models and design assumptions on the conduits and nanoprobes used to maintain the conduit and/or lessen uncertainty in pipeline integrity. In particular, the present techniques may include inputting corrosion and flow modeling to predict response to change in sulfate or other key metabolic components as indicated by rapid increase in H₂S resulting in pipeline fracture or cracking.

In one or more embodiments, the present techniques may include developing a model leading corrosion indicators by distinguishing abiotic biologic and/or combined corrosion sources. The distribution may enhance weight loss corrosion modeling based on the dominant modifier of pipe surface conditions and how rapidly these are to advance under certain conditions. For example, the present techniques may be utilized as part of a pipeline lifespan analyses. The process may consider consistent rate of pipe wall loss given particular compositional criteria, certain corrosion mechanisms, rate the corrosion over the life of the pipe and how these are subsequently modified depending on predicted treatment efficacy scenarios. The corrosion models may be designed assuming predictable general corrosion rates for the pipe surface as a whole. Localized sources of advanced corrosion may be outside of the model capability and hence, not part of the model predictions. Also, persistence of corrosion especially microbially influenced corrosion over time may likely increase pitting locally with pipe integrity consequences occurring earlier than anticipated.

In one or more embodiments, the probe composition (e.g., nanoprobe compositions) may be released into the conduit through different techniques. For example, the nanoprobe composition may be mixed into a carrier medium (e.g., fluid solution or suspension), for example in conduit fluid or compatible fluid with the conduit fluid, the mixture may be pumped into the conduit or into storage vessel upstream of the conduit.

In other embodiments, a probe composition may be attached, by any means known in the art, to a delivery device, which may be disposed into a conduit or otherwise made to traverse the conduit to a specific location. Then, the delivery device may be configured to activate and release the probes at a location upstream of the desired release location. In particular embodiments, the delivery probe may be attached to the monitoring tool or other suitable vehicle. In such embodiments, the monitoring tool or suitable vehicle may pass over a target location and then travel upstream to release the nanoprobes upstream of the target location. Then, the monitoring tool may travel to the target location in an attempt to detect any signals that may be generated by the signal generators of the nanoprobes or to verify the activities of the chemical treatment on the corrosive environment.

A specific advantage of the probes, methods, and systems is that each probe contains both a tag and one or more of a signal generator and biocide in a single package. As a result, the probes can begin producing usable signals directly from contact with the target of interest (in some embodiments, after being activated). This is particularly advantageous, especially in certain conduits, which are challenging to access and/or monitor.

It should be understood that the preceding is merely a detailed description of specific embodiments of the invention and that numerous changes, modifications, and alternatives to the disclosed embodiments can be made in accordance with the disclosure here without departing from the scope of the invention. The preceding description, therefore, is not meant to limit the scope of the invention. Rather, the scope of the invention is to be determined only by the appended claims and their equivalents. It is also contemplated that structures and features embodied in the present examples can be altered, rearranged, substituted, deleted, duplicated, combined, or added to each other. The articles “the”, “a” and “an” are not necessarily limited to mean only one, but rather are inclusive and open ended so as to include, optionally, multiple such elements.

What is claimed is:

1. A method of identifying and treating biologic materials of interest within a conduit comprising:
providing a probe composition comprising one or more probes;
wherein each of the one or more probes comprises:
(a) a tag; and
(b) one or more of a signal generator and a chemical treatment, wherein the probe is configured to generate a signal when the tag associates with a target biologic material, if the signal generator is present in the probe, and release the chemical treatment when the tag associates with the target biologic material, if the chemical treatment is present in the probe;
releasing the probes into a conduit;
if the probe composition includes one or more probes having the signal generator, detecting the presence of a signal generated by the signal generator on association of the tag with the target biologic material.

2. The method of claim 1, wherein the tag is one or more of a geomolecular tracer, an enzyme, a DNA primer, and a RNA primer.

3. The method of claim 1, wherein the signal generator is a nanoparticle.

4. The method of claim 1, wherein the signal generator is an inorganic fluorophore.

5. The method of claim 4, wherein the particle is one or more of a silicon nanoparticle, a mesoporous silica nanoparticle, a graphene quantum dot, a core/shell composite, a cadmium selenide nanoparticle, a cadmium-sulfide nanoparticle, a quantum dot, a nanoparticle composite, a nanocrystal, and a carbon nanotube.

6. The method of claim 1, wherein association of the tag with the target biologic material comprises one or more of sorbing, partitioning, ionic bonding, hydrogen bonding, adsorption, covalent bonding, adhesion, electrostatic interactions.

7. The method of claim 1, wherein the signal generated is at least one of an audible, a sonar, an acoustic, a visible, and a fluorescent signal.

8. The method of claim 1, wherein the tag is a DNA or RNA primer and the target is genetic material of the microorganisms that metabolize the geological, hydrocarbon or conduit material.

9. The method of claim 1, wherein detecting further comprises using one or more of a UV-Vis spectrometer, IR spectrometer, a fluorimeter, a Raman spectrometer, and a sonar detector.

10. The method of claim 1, wherein the one or more probes comprise nanoprobes.

11. A probe composition comprising:
one or more probes, wherein the probe comprises:
(a) at least one tag capable of associating with a target biologic material; and
(b) one or more of a signal generator and a chemical treatment, wherein the probe is configured to (i) generate a signal when the tag associates with a target biologic material, if a signal generator is present in the probe, and (ii) release the chemical treatment when the tag associates with the target biologic material, if a chemical treatment is present in the probe.

12. The probe composition of claim 11, further comprising a reagent.

13. The probe composition of claim 12, wherein the reagent is selected from the group consisting of water, brine, organic solvents, and a mixture thereof.

14. The probe composition of claim 11, wherein the tag is one or more of a geomolecular tracer, an enzyme, a DNA primer, and a RNA primer.

15. The probe composition of claim 11, wherein the signal generator is a nanoparticle.

16. The probe composition of claim 11, wherein the signal generator is an inorganic fluorophore.

17. The probe composition of claim 15, wherein the nanoparticle is one or more of a silicon nanoparticle, a mesoporous silica nanoparticle, a graphene quantum dot, a core/shell composite, a cadmium selenide nanoparticle, a cadmium-sulfide nanoparticle, a quantum dot, a nanoparticle composite, a nanocrystal, and a carbon nanotube.

18. The probe composition of claim 11, wherein the signal generated is at least one of an audible, a sonar, an acoustic, a visible, and a fluorescent signal.

19. The probe composition of claim 11, wherein the one or more probes comprise nanoprobes.

20. A method of identifying and treating biologic materials within a conduit comprising:
(a) providing a first probe;
wherein the first probe comprises:
(i) a first tag that associates with a first target biologic material; and
(ii) one or more of a first signal generator and a first chemical treatment, wherein the first probe is configured to generate a first signal when the first tag associates with the first target biologic material, if the first signal generator is present in the first probe, and release the first chemical treatment when the first tag associates with the first target biologic material, if the first chemical treatment is present in the first probe;
(b) providing a second probe;
wherein the second probe comprises:
(i) a second tag that associate with a second target biologic material; and
(ii) one or more of a second signal generator and a second chemical treatment, wherein the second probe is configured to generate a second signal when the second tag associates with the second target biologic material, if the second signal generator is present in the second probe, and release the second chemical treatment when the second tag associates with the second target biologic material, if the second chemical treatment is present in the second probe;
(c) releasing a probe composition comprising the first probe and the second probe into a conduit;
(d) if the probe composition includes a first probe having the first signal generator, measuring the first signal, wherein the first signal is generated by the first signal generator on association of the first tag with the first target biologic material; and
(e) if the probe composition includes a second probe having the second signal generator, measuring the second signal, wherein the second signal is generated by the second signal generator on association of the second tag with the second target biologic material.

21. The method of claim 20, wherein the first probe has a first signal generator and the second probe has a second signal generator and further comprising:
determining comparing the first signal to the second signal; and
deriving an estimation of the respective proportions of water and target in the target biological materials.

22. The method of claim 21, wherein the first signal and/or the second signal generated is at least one of an audible signal, a sonar signal, an acoustic signal, a visible signal, and a fluorescent signal.

23. The method of claim 20, wherein the one or more of the first tag and the second tag are hydrophilic.

24. The method of claim 20, wherein the probe composition further comprises a reagent.

25. The method of claim 21, wherein the reagent is selected from the group consisting of water, brine, organic solvents, and a mixture thereof.

26. The method of claim 20, wherein the one or more the first probe and second probe comprise nanoprobes.

27. A system for the identifying and treating biologic materials or corrosive environments within a conduit comprising:
(a) a delivery device configured to store a probe composition comprising one or more probes;
wherein each of the one or more probes comprises:
(i) a tag capable of associating with a target biologic material or corrosive environment; and
(ii) one or more of a signal generator, a biocide, wherein the probe is configured to generate a signal when the tag associates with a target biologic material or a corrosive environment, if a signal generator is present in the probe, release the biocide when the tag associates with the target biologic material, if a biocide is present in the probe; and release the corrosion inhibitor when the tag associates with the target corrosive environment, if an inhibitor is present in the probe;
and
(b) at least one detector capable of monitoring the interaction of the target biologic materials or corrosive environments with the one or more probes.

28. The system of claim 27, wherein the at least one detector comprises monitoring tool configured to be disposed within the conduit and configured to measure data within the conduit with one or more sensors.

29. The system of claim 27, wherein the one or more sensors are configured to detect a signal generated by the signal generator.

30. The system of claim 27, wherein the probe composition further comprises a reagent.

31. The system of claim 27, wherein the one or more probes comprise nanoprobes.

32. The system of claim 27, wherein the one or more sensor comprises one or more of a UV-Vis spectrometer, IR spectrometer, a fluorimeter, a Raman spectrometer, and a sonar detector.

33. A method of identifying and treating corrosive environments of interest within a conduit comprising:
providing a probe composition comprising one or more probes;
wherein each of the one or more probes comprises:
(c) a tag; and
(d) one or more of a signal generator and a chemical treatment, wherein the probe is configured to generate a signal when the tag associates with a target corrosive environment, if the signal generator is present in the probe, and release the chemical treatment when the tag associates with the target corrosive environment, if the chemical treatment is present in the probe;
releasing the probes into a conduit;
if the probe composition includes one or more probes having the signal generator, detecting the presence of a signal generated by the signal generator on association of the tag with the target corrosive environment.

34. The method of claim 33, wherein the tag is one or more of a redox sensitive tracer, reactive functional tracer, anode potential tracer, and cathode potential tracer.

35. The method of claim 33, wherein the signal generator is a nanoparticle.

36. The method of claim 33, wherein the signal generator is an inorganic fluorophore.

37. The method of claim 36, wherein the particle is one or more of a silicon nanoparticle, a mesoporous silica nanoparticle, a graphene quantum dot, a core/shell composite, a cadmium selenide nanoparticle, a cadmium-sulfide nanoparticle, a quantum dot, a nanoparticle composite, a nanocrystal, and a carbon nanotube.

38. The method of claim 33, wherein association of the tag with the target corrosive environment comprises one or more of sorbing, partitioning, ionic bonding, hydrogen bonding, adsorption, covalent bonding, adhesion, electrostatic interactions.

39. The method of claim 33, wherein the signal generated is at least one of an audible, a sonar, an acoustic, a visible, and a fluorescent signal.

40. The method of claim 33, wherein detecting further comprises using one or more of a UV-Vis spectrometer, IR spectrometer, a fluorimeter, a Raman spectrometer, and a sonar detector.

41. The method of claim 33, wherein the one or more probes comprise nanoprobes.
